(19) United States
(12) Patent Application Publication
(10) Pub. No.: US 2018/0289708 A1
(43) Pub. Date: Oct. 11, 2018

(54) Low dose oral dipyridamole compositions and uses thereof

(71) Applicant: Moshe ROGOSNITZKY, Kiryat Yearim (IL)

(72) Inventor: Moshe ROGOSNITZKY, Kiryat Yearim (IL)

(21) Appl. No.: 15/766,881

(22) PCT Filed: Oct. 14, 2016
(86) PCT No.: PCT/IB2016/056151

§ 371 Date: Apr. 9, 2018

Related U.S. Application Data
(60) Provisional application No. 62/325,516, filed on Apr. 21, 2016.
(60) Provisional application No. 62/241,771, filed on Oct. 15, 2015.

(51) Int. Cl. A61K 31/519; A61K 9/00; A61P 15/10; A61P 25/20; A61P 27/04; A61P 29/02; A61P 31/12

(52) U.S. Cl. CPC A61K 31/519; A61K 9/0053; A61P 15/10; A61P 25/20; A61K 31/4045; A61P 29/02; A61P 31/12; A61K 2300/00; A61P 27/04

(57) ABSTRACT

A composition comprising about 10 mg or less of dipyridamole formulated for oral administration and a physiologically acceptable carrier.

### RELATED APPLICATIONS

This application claims the benefit under 35 U.S.C. § 119(e) of provisional application U.S. 62/241,771 filed 15 Oct. 2015 and entitled “Low dose oral dipyridamole compositions and uses thereof”; and

This application claims the benefit under 35 U.S.C. § 119(e) of provisional application U.S. 62/325,516 filed 21 Apr. 2016 and entitled “Low dose oral dipyridamole compositions and uses thereof;

Each of which is fully incorporated herein by reference.

### FIELD OF THE INVENTION

Various described embodiments relate to oral dosage forms of dipyridamole and their preparation and use.

### BACKGROUND OF THE INVENTION

Oral dipyridamole is currently marketed as 25, 50, 75 and 200 mg tablets (by numerous manufacturers (for example Boehringer Ingelheim as PERSANTINE®) for use in preventing blood clots after heart valve surgery. Dipyridamole is available in a liquid oral formulation (Rosemount Pharmaceuticals, UK) as a 50 mg/5 mL suspension for use in preventing formation of blood clots with a recommended 300-600 mg/day dosage. Dipyridamole is also available as 25 mg dragees or tablets under trademark CURANTYL® (Berlin-Chemie AG/Menarini Group) and is approved in Russia for reduction of platelet aggregation (75-225 mg up to 600 mg/day dose), prevention of influenza and other acute respiratory viral infections (50 mg/day dosage, 1 times a week for 4-5 weeks) and respiratory viral infections in ill patients (2×25 mg/day dosage, with dosage interval of minimum 2 hours apart, once a week for 8-10 weeks).

Dipyridamole is absorbed from the gastrointestinal tract, reaching peak plasma levels in humans 1-3 hours following oral administration. Peak plasma levels are dose dependent.

Geometric mean (range) peak plasma concentrations at steady state conditions with 75 mg t.d.s. were 1.86 μg/mL (1.23-3.27 μg/mL), and at trough were 0.13 μg/mL (0.06-0.26 μg/mL).

With 75 mg q.i.d. corresponding peak concentrations were 1.54 μg/mL (0.975-2.17 μg/mL), trough concentrations were 0.269 μg/mL (0.168-0.547 μg/mL).

With 100 mg q.i.d. corresponding peak concentrations were 2.36 μg/mL (1.13-3.81 μg/mL), trough concentrations were 0.432 μg/mL (0.186-1.38 μg/mL).

The dose linearity of dipyridamole after single dose administration was demonstrated in the range from 25 to 150 mg. Blood levels are quite variable, possibly to depending on food intake, individual stomach acid levels and gastrointestinal peristalsis. Ingestion on an empty stomach may result in higher blood levels. Following intravenous (IV) administration, the distribution half-life in humans is about 25 minutes, and after oral administration, is about 3 hours. When plasma levels of drug are followed for up to 60 hours after IV or oral administration of 20-50 mg, plasma levels decline tri-exponentially with half-lives of 5 minutes (IV only), 53 minutes, and about 10-12 hours. The volume of distribution is about 140 L with about 92 to 99% binding to plasma proteins, primarily alpha1-acid glycoprotein.

Daily oral doses of dipyridamole often range from 100-400 mg.

The common cold (also known as nasopharyngitis, rhinopharyngitis, acute coryza, head cold, or simply a cold) is a viral infectious disease of the upper respiratory tract which primarily affects the nose. Signs and symptoms of a cold include coughing, sore throat, runny nose, sneezing, and fever which usually resolve in seven to ten days, with some symptoms lasting up to three weeks. Well over 200 virus strains are implicated in the cause of the common cold; the rhinoviruses are the most common.

Erectile dysfunction in males is a debilitating condition which negatively impacts on the psychological, sexual and social wellbeing of the individual. For decades the presence or lack of morning erections upon awakening have been used to diagnose physiological erectile dysfunction. It has been suggested that this may also reflect a lack of testosterone production. Erectile dysfunction can originate from underlying psychological or physiological conditions. There is close correlation between erectile dysfunction (indicating penile artery disease) and blocked arteries around the heart. Erection problems are often a first sign of diabetes and an early sign of multiple sclerosis.

Anxiety and anxiety disorders prevent the sufferer from performing everyday activities and tasks, due to some perceived concern, fear, worry or other external stimulus. Consequently anxiety can have a severe negative impact on the wellbeing of the subject.

Hair loss can occur in both male and female humans due to a variety of underlying reasons. Both female pattern baldness and male pattern baldness affect millions of individuals globally with potential negative social and psychological impact. Only two drugs have been approved by the FDA as treatments for androgenic alopecia: minidoxil (ROGAINE® and finasteride (PROPECIA)®/PROSCAR®, the latter has been reported to lead to permanent sexual side effects such as low libido, erectile dysfunction and decreased arousal in a high number of subjects even after discontinuation of the treatment. Hair loss can also involve loss of or lack of growth of bodily hair, including eye lashes and eye brows.

“Dry eye” is a disorder of the tear film due to tear deficiency or excessive tear evaporation which causes damage to the interpalpebral ocular surface and is associated with symptoms of ocular discomfort. Dry eye includes two major classes: (i) aqueous tear deficient dry eye (ADDE), and (ii) evaporative dry eye (EDE). Dry eye is also referred to as keratoconjunctivitis sicca.

ADDE refers mainly to a failure of sufficient tear secretion due to lacrimal dysfunction. ADDE has two major subclasses: (i) Sjorgen's Syndrome dry eye (SSDE), and (ii) non-SS dry eye (such as in Graft-versus-Host Disease (GvHD) or in diabetes mellitus or allergy-induced dry eye, or non-specific dry eye).

Meibomian Gland Dysfunction and Graft Vs Host Disease (GvHD) are other causes of dry eye disease and ocular surface inflammation.

EDE may be: (i) intrinsic, due to diseases affecting lid structures or dynamics, or (ii) extrinsic, in which ocular surface disease occurs due to some extrinsic exposure, such as topical drug preservatives, contact lens wear, pterygium, pinguecula, exposure to air conditioned environments, or vitamin A deficiency. The use of topically administered dipyridamole for treatment of dry eye and other eye disorders is taught by Rogosnitzky in U.S. Pat. No. 9,254,289.

Xerostomia (“dry mouth”) is a disorder arising due to reduced salivary flow from the salivary glands and results in the perception by an individual of a “dry mouth”. Xerostomia can result in a range of symptoms such as halitosis, oral soreness and burning, difficulty swallowing and talking, and altered taste and result in the increase in dental decay. The incidence of xerostomia is reported to range from 11-47% dependent on population demographics. Dry mouth can occur in an individual for a variety of reasons including Sjögren's syndrome, HIV/AIDS, Alzheimer's disease, diabetes, anemia, rheumatoid arthritis and hypertension for instance.

Xerostomia and dry eye disease often occur together in the same individuals. They are not necessarily but can be etiologically connected.

Angina is a term used for chest pain caused by reduced blood flow to the heart muscle. Angina is a symptom of coronary artery disease. Angina is typically described as squeezing, pressure, heaviness, tightness or pain in one's chest. Angina, also called angina pectoris, can be a recurring problem or a sudden, acute health concern. Angina pain can be caused by many things. Often the pain is chronic. Sometimes patients with pain become dependent on opiate drugs for adequate pain relief. However, these drugs have many side effects. In some cases Angina pain is chronic rheumatic type.

Insomnia is a sleep disorder in which there is an inability to fall asleep or to stay asleep as long as desired. While the term is sometimes used to describe a disorder demonstrated by polysomnographic or actigraphic evidence of disturbed sleep, insomnia is often practically defined as a positive response to either of two questions: “Do you experience difficulty sleeping?” or “Do you have difficulty falling or staying asleep?” Insomnia is most often thought of as both a medical sign and a symptom that can accompany several sleep, medical, and psychiatric disorders characterized by a persistent difficulty falling asleep and/or staying asleep and/or sleep of poor quality. Insomnia is typically followed by functional impairment while awake. Insomnia can occur at any age, but it is particularly common in the elderly. Insomnia can be short term (up to three weeks) or long term (above 3-4 weeks).

Insomnia can lead to memory problems, depression, irritability and an increased risk of heart disease and/or automobile related accidents. Insomnia can be grouped into primary and secondary, or comorbid, insomnia.

Primary insomnia is a sleep disorder not attributable to a medical, psychiatric, or environmental cause. It is described as a complaint of prolonged sleep onset latency, disturbance of sleep maintenance, or the experience of non-refreshing sleep. A complete diagnosis will differentiate between free-standing primary insomnia, insomnia as secondary to another condition, and primary insomnia co-morbid with one or more conditions.

Insomnia is estimated to affect approximately 25%-50 of the human population for some duration during their lives and manifests itself in individuals as difficulty falling asleep, poor quality or duration of sleep and non-restorative sleep. Insomnia can also present alongside other conditions such as pain. The resultant loss of sleep for the individual naturally reduces quality of life, increases stress and risk of accidents.

Cognitive behavioral therapy is useful in insomnia that is present for a long duration. Those who are having trouble sleeping sometimes turn to sleeping pills, which may help, but also may lead to substance dependency or addiction if used regularly for an extended period.

B-cell chronic lymphocytic leukemia (B-CLL), also known as chronic lymphoid leukemia (CLL), is the most common type of leukemia (a type of cancer of the white blood cells) in adults. CLL affects B cell lymphocytes, which originate in the bone marrow, develop in the lymph nodes, and normally fight infection by producing antibodies.

In CLL, B cells grow out of control and accumulate in the bone marrow and blood, where they crowd out healthy blood cells. CLL is a stage of small lymphocytic lymphoma (SLL), a type of B-cell lymphoma, which presents primarily in the lymph nodes. CLL and SLL are considered the same underlying disease, just with different appearances.

CLL is primarily a disease of adults. Most (>75%) people newly diagnosed with CLL are over the age of 50, and the majority are men. However, in rare cases, it can occur in teenagers and occasionally in children. Some of these may relate to an inherited predisposition.

Most people are diagnosed without symptoms as the result of a routine blood test that returns a high white blood cell count, but, as it advances, CLL results in swollen lymph nodes, spleen, and liver, and eventually anemia and infections. Early CLL is currently not treated, and late CLL is currently treated with chemotherapy and monoclonal antibodies.

Prostate cancer is a type of tumor that affects the prostate gland, and often spreads to the bones and other organs. It is usually diagnosed when Prostate Specific Antigen (PSA) levels in the blood rise above normal. Sometimes it is diagnosed when the patient is already symptomatic. Common medical practice in many countries is to adopt a “wait and watch” approach in patients and keep a close eye on the rate of rise in PSA levels. Reduction of PSA levels is typically interpreted as successful response to treatment.

### SUMMARY OF THE INVENTION

One aspect of some embodiments of the invention relates to low dose solid oral dosage forms containing ≤10 mg dipyridamole per dosage unit and their use in treatment of a range of indications at a daily dose of one or two units per day.

More specifically some embodiments of the invention relates to the use of the low dose oral dosage forms of dipyridamole in the treatment of conditions such as viral infections (e.g. common cold with or without sore throat, SARS, influenza. ZIKA virus) and/or erectile dysfunction and/or sleep disorders (such as insomnia, lack of REM sleep, vivid dreams) and/or anxiety and/or pain, and/or opiate addiction and/or xerostomia and/or dry eye disease and/or hair loss and/or prostate cancer and/or chronic lymphocytic leukemia and/or GvHD.

There is currently no marketed oral solid dosage form of dipyridamole at 10 mg, or lower per unit. Applicant is unaware of any reports on the use of low oral dosage dipyridamole across a range of therapeutic indications such viral infections (such as common cold with sore throat, SARS, influenza), erectile dysfunction, sleep disorders (such as insomnia, lack of REM sleep, vivid dreams), anxiety, pain, pain treated with opiates, xerostomia, hair loss, cataracts, dry eye, dry eye disease, immune regulation, allergies, allergic rhinitis, allergic conjunctivitis, angina, Crohn's disease or any other indication.

For purposes of this specification and the accompanying claims the term “low dosage” or “low dose” indicates equal to or less than about 20 mg/day. In some embodiments a dose of one or two units per day is used for treatment. In some embodiments dipyridamole is the sole active ingredient present in a physiologically effective amount. Alternatively or additionally, in various embodiments the daily dose is provided as one, two, three, four or five doses.

According to various exemplary embodiments of the invention the total daily dose of dipyridamole is 0.25 mg, 0.5 mg, 1.0 mg, 2 mg, 3 mg, 4 mg, 5 mg, 6 mg, 7 mg, 8 mg, 9 mg, 10 mg or 20 mg/day or intermediate or lesser amounts.

According to various exemplary embodiments of the invention the daily dose is delivered as one, two or three oral dosage forms/day.

According to various exemplary embodiments of the invention the size of an oral dosage form is 0.125 mg, 0.25 mg, 0.5 mg, 1.0 mg, 2 mg, 3 mg, 4 mg, 5 mg, 6 mg, 7 mg, 8 mg, 9 mg or 10 mg or intermediate or lesser amounts.

For purposes of this specification and the accompanying claims the term “about” indicates +/−10%.

Oral compositions include both liquid and solid dosage forms such as capsules, dragees, tablets and solution and/or suspensions prepared with pharmaceutically acceptable excipients and/or carriers.

Another aspect of some embodiments of the invention relates to treatment packs containing oral dosage forms with ≤10 mg of dipyridamole, packaging materials and instructions specifying a suitable dosage regimen for treatment of one or more indications disclosed herein.

Such oral dosage forms and treatment packs solve technical problems associated with treatment of such ailments. Alternatively or additionally, such oral dosage forms and treatment kits overcome technical problems associated with side effects from use of dipyridamole at previously recommended higher dosages.

It will be appreciated that the various aspects described above relate to solution of technical problems associated with the treatment of conditions such as viral infections (such as common cold with or without sore throat, SARS, influenza), erectile dysfunction, sleep disorders (such as insomnia, lack of REM sleep, vivid dreams), anxiety, pain, pain treated with opiates, xerostomia, dry eye disease, hair loss and cancer treatment.

Another aspect of some embodiments of the invention relates to administration of low doses of dipyridamole via alternative routes. Alternative routes include buccal administration, and transdermal administration. Transdermal administration includes administration as a topical cream, ointment, gel or slow release patch wherein the transdermal formulation is not designed for topical treatment but rather as a means for systemic delivery via absorption through the skin.

Alternatively or additionally, it will be appreciated that the various aspects described above relate to solution of technical problems related to application of a known active ingredient to new clinical uses.

In some exemplary embodiments of the invention there is provided a composition including: (a) a physiologically effective amount of dipyridamole at low dosage of about 10 mg or less per dosage unit and (b) a physiologically acceptable carrier. In some embodiments, the composition is formulated as a solution or suspension. In other exemplary embodiments of the invention, the composition is formulated as a solid dosage form. Alternatively or additionally, in some embodiments the composition is formulated as a liquid dosage form comprising propylene glycol. Alternatively or additionally, in some embodiments the composition is formulated as a soluble liquid dosage form with a concentration of 2 mg/ml or less. Alternatively or additionally, in some embodiments the composition is used in the treatment of viral infections, erectile dysfunction, sleep disorders, anxiety, pain, xerostomia, dry eye, prostate cancer, GvHD, chronic lymphocytic leukemia and/or hair loss. Alternatively or additionally, in some embodiments the composition includes melatonin.

In some exemplary embodiments of the invention there is provided a treatment pack including: (a) multiple doses of a composition containing about 10 mg or less per dosage unit dipyridamole as an active ingredient; (b) packaging material; and (c) instructions for oral administration of said composition to an individual at a daily dose of 20 mg or less.

Alternatively or additionally, in some embodiments the composition contains dipyridamole at a concentration of at least about 10⁻⁶ molar (moles/liter).

Alternatively or additionally, in some embodiments the treatment pack includes a single container for the multiple doses. Alternatively or additionally, in some embodiments the treatment pack includes multiple containers, each of the multiple containers containing a single dose of the multiple doses. Alternatively or additionally, in some embodiments the instructions identify the kit as useful in treatment of at least one member of the group consisting of viral infections, erectile dysfunction, sleep disorders, anxiety, pain, xerostomia, dry eye, keratoconjunctivitis sicca, cancer and hair loss. Alternatively or additionally, in some embodiments the composition is formulated in a form selected from the group consisting of solutions or solid oral dosage form. In some embodiments the solid oral dosage form is a capsule or tablet. Alternatively or additionally, in some embodiments the instructions specify a dosing regimen of one or two dosage forms per day or every other day.

In some exemplary embodiments of the invention there is provided a method including: identifying a subject in need of treatment for a disorder selected from the group consisting of insomnia, keratoconjunctivitis sicca, angina pectoris, non-specific recurrent sore throat, erectile dysfunction, chronic lymphocytic leukemia, viral disease, cancer-related bone pain, rheumatic or fibromyalgia pain, xerostomia, androgenetic alopecia and prostate cancer; and orally administering about 10 mg or less of dipyridamole per day to the subject. In some embodiments the method includes orally administering melatonin to the subject. Alternatively or additionally, in some embodiments the disorder is insomnia. Alternatively or additionally, in some embodiments the disorder is keratoconjunctivitis sicca. Alternatively or additionally, in some embodiments the disorder is angina pectoris. Alternatively or additionally, in some embodiments the disorder is non-specific recurrent sore throat. Alternatively or additionally, in some embodiments the disorder is erectile dysfunction. Alternatively or additionally, in some embodiments the disorder is chronic lymphocytic leukemia. Alternatively or additionally, in some embodiments the disorder is viral disease. Alternatively or additionally, in some embodiments the disorder is cancer-related bone pain. Alternatively or additionally, in some embodiments the disorder is rheumatic or fibromyalgia pain. Alternatively or additionally, in some embodiments the disorder is xerostomia. Alternatively or additionally, in some embodiments the disorder is androgenetic alopecia. Alternatively or additionally, in some embodiments the disorder is prostate cancer.

Some exemplary embodiments of the invention relate to use of 10 mg or less of dipyridamole (e.g. as an oral dosage form) in the treatment of a disorder selected from the group consisting of insomnia, keratoconjunctivitis sicca, angina pectoris, non-specific recurrent sore throat, erectile dysfunction, chronic lymphocytic leukemia, viral disease, cancer-related bone pain, rheumatic or fibromyalgia pain, xerostomia, androgenetic alopecia and prostate cancer. In some embodiments melatonin is included in the oral dosage form. Alternatively or additionally, in some embodiments the disorder is insomnia. Alternatively or additionally, in some embodiments the disorder is keratoconjunctivitis sicca. Alternatively or additionally, in some embodiments the disorder is angina pectoris. Alternatively or additionally, in some embodiments the disorder is non-specific recurrent sore throat. Alternatively or additionally, in some embodiments the disorder is erectile dysfunction. Alternatively or additionally, in some embodiments the disorder is chronic lymphocytic leukemia. Alternatively or additionally, in some embodiments the disorder is viral disease. Alternatively or additionally, in some embodiments the disorder is cancer-related bone pain. Alternatively or additionally, in some embodiments the disorder is rheumatic or fibromyalgia pain. Alternatively or additionally, in some embodiments the disorder is xerostomia. Alternatively or additionally, in some embodiments the disorder is androgenetic alopecia. Alternatively or additionally, in some embodiments is prostate cancer.

According to one aspect of some embodiments of the invention the therapeutic effects observed with low dose(s) of dipyridamole diminish with increasing doses. Thus, to despite the fact that substantially higher dosage regimens (up to 400 mg daily) of dipyridamole are reported in the literature, the instantly disclosed therapeutic benefits have not been observed before. Alternatively or additionally, the instantly disclosed low doses of dipyridamole contribute to a reduction in side effects commonly reported for higher doses of dipyridamole, such as headaches.

While high doses of dipyridamole have been proposed to induce interferon production as an anti-viral treatment, the instantly disclosed low doses produce superior anti-viral effects but with less side effects than the normal (higher) dose.

According to one aspect of some embodiments of the invention dipyridamole combined with melatonin induces sleep more effectively than either dipyridamole or melatonin on their own. The sleep inducing effect appears to be synergistic.

Unless otherwise defined, all technical and scientific terms used herein have the same meaning as commonly understood by one of ordinary skill in the art to which this invention belongs. Although suitable methods and materials are described below, methods and materials similar or equivalent to those described herein can be used in the practice of the present invention. In case of conflict, the patent specification, including definitions, will control. All materials, methods, and examples are illustrative only and are not intended to be limiting.

As used herein, the terms “comprising” and “including” or grammatical variants thereof are to be taken as specifying inclusion of the stated features, actions or components without precluding the addition of one or more additional features, actions, components or groups thereof. This term is broader than, and includes the terms “consisting of” and “consisting essentially of” as defined by the Manual of Patent Examination Procedure of the United States Patent and Trademark Office. Thus, any recitation that an embodiment “includes” or “comprises” a feature is a specific statement that sub embodiments “consist essentially of” and/or “consist of” the recited feature.

The term “method” refers to manners, means, techniques and procedures for accomplishing a given task including, but not limited to, those manners, means, techniques and procedures either known to, or readily developed from known manners, means, techniques and procedures by practitioners of architecture and/or computer science.

### DETAILED DESCRIPTION OF EMBODIMENTS

Embodiments of the invention relate to therapeutic compositions for oral administration containing about 10 mg or less per dosage unit dipyridamole and treatment packs including such compositions and methods for producing such treatment packs and/or compositions.

Specifically, some embodiments of the invention can be used to treat various ailments while reducing the likelihood of undesirable side effects.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

### Exemplary Compositions

Some embodiments of the invention relate to pharmaceutical composition. According to various exemplary embodiments of the invention the pharmaceutical composition includes 10 mg, 7.5 mg, 5 mg, 2.5 mg, 1 mg, 0.5 mg or 0.25 mg, or lesser or intermediate amounts of dipyridamole formulated for oral administration and a physiologically acceptable carrier. In some embodiments the pharmaceutical composition is formulated as a liquid dosage form dissolved with propylene glycol. In some embodiments propylene glycol is the primary inactive ingredient in the composition. Alternatively or additionally, in some embodiments the liquid dosage form has a concentration of 2 mg/ml or less. In some embodiments the composition is formulated as a solid dosage form (e.g. pill, tablet or capsule). In some embodiments the composition is provided as a liquid filled capsule.

### Exemplary Treatment Methods

According to various exemplary embodiments of the invention, compositions described above are administered to a subject in need thereof 1 or 2 or 3 or 4 or 5 or more times daily to achieve a total daily dose of 1, 5, 10, 15 or 20 mg/day or lesser or intermediate total daily dose. In some embodiments the compositions described above are administered once every other day. Subjects in need thereof suffer from viral infections and/or erectile dysfunction and/or sleep disorders and/or anxiety and/or pain and/or xerostomia and/or hair loss and/or dry eye and/or cancer, and/or dry eye.

### Exemplary Treatment Packs

Some exemplary embodiments of the invention relate to a treatment pack including multiple oral dosage forms of a composition containing dipyridamole as an active ingredient. In some embodiments each oral dosage form contains 10 mg, 7.5 mg, 5.0 mg, 2.5 mg. 1.0 mg or 0.5 mg or intermediate or smaller amounts of dipyridamole. The treatment pack also includes packaging material and instructions for oral administration of the composition with a total daily dose of 20 mg/day, 10 mg/day, 5 mg/day, 2.5 mg/day, 1.0 mg/day, 0.5 mg/day, 0.25 mg/day or intermediate or lower daily dosages. According to various exemplary embodiments of the invention instructions included in the kit indicate that the product is useful in treatment of viral infections and/or erectile dysfunction and/or sleep disorders and/or anxiety and/or pain and/or xerostomia and/or hair loss and/or dry eye and/or prostate cancer and/or chronic lymphocytic leukemia. According to various exemplary embodiments of the invention the composition in the kit is formulated as described hereinabove under “Exemplary Compositions”. Alternatively or additionally, in some embodiments the instructions in the kit specify a dosing regimen of one or two dosage forms per day or every other day. In some embodiments the kit includes a single container for the multiple doses (e.g. a bottle of syrup or elixir or a single bottle containing multiple solid oral dosage forms). In other exemplary embodiments of the invention, the kit includes multiple containers, each of the multiple containers containing a single dose of the multiple doses (e.g. individual containers with pre-measured liquid doses or individual solid dosage forms in bubble packs, blister packs or foil pack wrapping).

### Exemplary Dosages

According to various exemplary embodiments of the invention the total daily dose of dipyridamole is 0.25 mg, 0.5 mg, 1 mg, 2 mg, 3 mg, 4 mg, 5 mg, 6 mg, 7 mg, 8 mg, 9 mg, 10 mg or 20 mg/day or intermediate or lesser amounts. According to various exemplary embodiments of the invention the daily dose is given in 1, 2, 3, 4 or 5 doses at least every other day. According to various exemplary embodiments of the invention oral dosage forms such as tablets, capsules, gel-caps, dragees, solutions and suspensions are employed.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

Specifically, a variety of numerical indicators have been utilized. It should be understood that these numerical indicators could vary even further based upon a variety of engineering principles, materials, intended use and designs incorporated into the various embodiments of the invention. Additionally, components and/or actions ascribed to exemplary embodiments of the invention and depicted as a single unit may be divided into subunits. Conversely, components and/or actions ascribed to exemplary embodiments of the invention and depicted as sub-units/individual actions may be combined into a single unit/action with the described/depicted function.

Alternatively, or additionally, features used to describe a method can be used to characterize an apparatus and features used to describe an apparatus can be used to characterize a method.

It should be further understood that the individual features described hereinabove can be combined in all possible combinations and sub-combinations to produce additional embodiments of the invention. The examples given above are exemplary in nature and are not intended to limit the scope of the invention which is defined solely by the following claims.

Each recitation of an embodiment of the invention that includes a specific feature, part, component, module or process is an explicit statement that additional embodiments of the invention not including the recited feature, part, component, module or process exist.

All publications, references, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “include”, and “have” and their conjugates as used herein mean “including but not necessarily limited to”.

### Exemplary Physiologic Considerations

Dipyridamole absorption is believed to be dependent on physiologic conditions (e.g. pH levels i.e. increased absorption at more acidic levels) and/or patient's age and/or fasting status. It is therefore anticipated dosage forms which increase absorption efficiency and/or absorption rate will lead to efficacy of lower doses.

Additional objects, advantages, and novel features of various embodiments of the invention will become apparent to one ordinarily skilled in the art upon examination of the following examples, which are not limiting. Additionally, each of the various embodiments and aspects of the present invention as delineated hereinabove and as claimed in the claims section below finds experimental support in the following examples.

Despite the fact that dipyridamole is difficult to dissolve in water, it was surprisingly found to be soluble in propylene glycol, at a concentration of 5 mg or 10 mg or more per ml.

### EXAMPLES

Reference is now made to the following examples, which together with the above descriptions, illustrate the invention in a non-limiting fashion.

### Example 1

### Preparation of Solid Oral Dosage Forms

Dipyridamole capsules were prepared as follows. Fish or beef gelatin capsules with 200 mg capacity were used. Dipyridamole powder was mixed into dextrose monohydrate powder as follows:

190 mg dextrose monohydrate+10 mg dipyridamole

195 mg dextrose monohydrate+5 mg dipyridamole

198 mg dextrose monohydrate+2 mg dipyridamole

199 mg dextrose monohydrate+1 mg dipyridamole

199.5 mg dextrose monohydrate+0.5 mg dipyridamole

199.75 mg dextrose monohydrate+0.25 mg dipyridamole

After this, capsules were filled and closed, and provided to users.

In other exemplary embodiments of the invention, rice powder or microcrystalline cellulose powder or Avicel or leucine is substituted for dextrose monohydrate.

### Example 2

### Preparation of Liquid Oral Dosage Forms

For some users who preferred a liquid formulation, dipyridamole was dissolved in pure propylene glycol to provide concentrations of 0.25 mg per drop (5 mg/mL), 0.5 mg per drop (10 mg/mL), and 1 mg per drop (20 mg/mL). Dipyridamole was surprisingly soluble in propylene glycol and maintained its potency even after 1 year of storage at room temperature. Solubility of a compound is known to increase absorption and efficacy. In other exemplary embodiments of the invention, liquid formulations are provided in gel-caps.

### Example 3

### Insomnia Treatment

In order to determine the effect of low dose oral dipyridamole for treating insomnia, 4 human subjects with intractable insomnia (who had failed numerous other treatments with different types of sleeping pills) were treated with low dose oral dipyridamole prepared as in example 1 as the sole treatment.

Subject 1 (48 y/o [year old] male), Subject 2 (22 y/o female), Subject 3 (13 y/o female) and Subject 4 (38 y/o female) were treated with varying doses (0.25, 0.5, 1, 2, 4, 5, 7 and 10 mg) taken before sleep, for periods of 2 weeks or more for each dose and followed for up to 7 months in total.

Subject 1: 2 mg was found to be the optimal dose for ensuring an excellent night's sleep. Additional effects included intense dreams, especially pleasant ones, dreams involving remote events and increased incidence of nocturnal penile tumescence which had been very rarely experienced in the past. Subject tried half a dozen sleep meds prior to this with either poor effect or unbearable side effects. Dipyridamole 10 mg had negligible benefit compared to lower doses.

Subject 2: 2 mg was found to be the optimal dose for ensuring an excellent, undisturbed sleep lasting approximately 6-8 hours per night. An additional effect included good dream recall. She previously used melatonin both regular and timed release without clear benefit.

Subject 3: 1 mg was found to be the optimal dose for ensuring an excellent, deep sleep. An additional effect included frightening dreams. The subject weighed 37 kg. Hence, a lower dose was used. She needed to take it by mid-evening in order to fall asleep easily.

Subject 4: 5 mg was found to be the optimal dose for ensuring ease in falling asleep and having deep restorative sleep. Additional effects included better dream recall and better mood. The subject had a diminishing effect with increasing dose.

This example illustrates that low dose oral dipyridamole is effective for the treatment of insomnia, even in patients that do not respond to conventional sleeping medications. This example also illustrates that the benefits are lost with increasing dosage, especially 10 mg or more which has little or no effect.

### Example 4

### Keratoconjunctivitis Sicca Treatment

In order to determine the effect of low dose oral dipyridamole for treating keratoconjunctivitis sicca, three subjects with keratoconjunctivitis sicca with duration between 3 & 12 years were treated with low dose oral dipyridamole prepared as in Example 1 as the sole treatment. Each of the three subjects was unresponsive to cyclosporine eye drops or to lubricating drops which are the conventional treatments.

Subject 1 (58 y/o female), Subject 2 (40 y/o male) and Subject 3 (40 y/o male) were treated with varying doses (2, 5 and 10 mg) taken before sleep or in the afternoon/evening hours, for initial periods of up to 2 weeks for each dose to establish optimal dosing, and followed for up to 12 months in total.

Subject 1: 2 mg was found to be the optimal dose for relieving the effects of keratoconjunctivitis sicca. She reported complete relief of dry eye symptoms (grittiness, graininess, itching), cessation of eye discharge, and was finally able to use a computer screen for a full day's work without symptoms. She was able to cease the copious use of lubricating drops. Additional effects included significantly improved quality of sleep and a great improvement in arthritis symptoms. For a period of 1 month, dipyridamole was prepared as in Example 2.

Subject 2: 2 mg was found to be the optimal dose which resulted in complete resolution of previous symptoms of burning, itching, grittiness and discomfort, and copious eye discharge. He no longer required lubricating eye drops or artificial tears. For a period of 1 month, dipyridamole was prepared as in Example 2. Additional effects included improved memory and sleep quality.

Subject 3: 2 mg was found to be the optimal dose for the resolution of symptoms and, normalization of tear osmolality and Schirmer's test which no longer showed evidence of dry eye. He was able to stop the use of lubricating eye drops, and artificial tears. The 10 mg dose was not effective and the 2 mg worked better than 5 mg. Additional effects included improved sleep and mood.

This example illustrates that low dose oral dipyridamole is effective for the treatment of keratoconjunctivitis sicca. The results also suggest that 2 mg/day is an effective dose.

### Example 5

### Angina Pectoris Treatment

In order to determine the effect of low dose oral dipyridamole for treating angina pectoris, 2 subjects with chronic treatment-resistant angina were treated with low dose oral dipyridamole as the sole treatment.

Subject 1 (74 y/o male) and Subject 2 (58 y/o male) were treated with varying doses of oral dipyridamole prepared as in Example 1 (1, 2, 3, 5 and 10 mg) taken in the morning or evening, for periods of up to 2 weeks for each dose and followed for up to 7 months in total.

Subject 1: 3 mg was found to be the optimal dose for complete resolution of angina. The 10 mg dose was ineffective. Additional effects included improved hair growth including eyelashes and eyebrows, and improved sleep.

Subject 2: 2 mg was found to be the optimal dose for complete resolution of angina symptoms (chest pain, shortness of breath). For a period of 1 month, dipyridamole was prepared as in Example 2. Additional effects included improved sleep and vivid dreams.

This example illustrates that low dose oral dipyridamole is effective for the treatment of angina pectoris, even in patients that do not respond to conventional treatments.

### Example 6

### Treatment of Non-specific Recurrent Sore Throat

In order to determine the effect of low dose oral dipyridamole for treating recurrent sore throat, one subject with recurrent, non-streptococcal, non-specific, sore throat occurring every 4-8 weeks and lasting 7-10 days was treated with low dose oral dipyridamole prepared as in example 1 as the sole treatment.

A 10 y/o female was treated with 1 mg taken before sleep and followed for 11 months in total. A dose of 1 mg/day resulted in complete resolution of recurrent sore throat. One mg dose was given based on a weight of 29 kg. An additional effect was the resolution of post nasal drip.

This example illustrates that low dose oral dipyridamole is effective for the treatment of recurrent sore throat.

### Example 7

### Erectile Dysfunction Treatment

In order to determine the effect of low dose oral dipyridamole for treating erectile dysfunction, 5 subjects with erectile dysfunction were treated with low dose oral dipyridamole prepared as in Example 1 as the sole treatment. Some subjects had either failed or could not tolerate conventional treatments.

Subject 1 (41 y/o male), Subject 2 (27 y/o male), Subject 3 (67 y/o male), Subject 4 (70 y/o male) and subject 5 (48 y/or male) were treated with varying doses (2, 3, 5 and 10 mg) taken in the evening or before sleep, for periods 2 weeks or more for each dose and followed for up to 15 months of use in total.

Subject 1: 2 mg was found to be the optimal dose for complete restoration of erectile function that had not been present for 6 years including absence of nocturnal penile tumescence for 12 years. The subject could not take sildenafil or similar agents due to adverse side effects. A consistent daily dose of dipyridamole was taken at night on a continuous basis. Nocturnal penile tumescence effect was experienced sooner (noticed if waking up in middle of the night) if taken on empty stomach, but always upon awakening in the morning. Additional effects included excellent dream recall, vivid dreams and improved sleep.

Subject 2: 3 mg was found to be the optimal dose for obtaining satisfactory erectile function. Subject 2 had not been able to achieve an erection for 3 years without the use of sildenafil, tadalafil or vardenafil. An additional effect included improved sleep.

Subject 3: Erectile function was restored to user's satisfaction within 10 days of starting dipyridamole at 2 mg. For a period of time, the subject increased dose to twice daily, still with satisfactory outcome. When the subject increased dose to 10 mg daily, the beneficial effect was lost.

Subject 4: Resolution of erectile dysfunction was achieved within 5 days of starting low dose oral dipyridamole at 2 mg. As the subject was satisfied with the result, no dose adjustment was attempted. The subject noticed increased resistance to upper respiratory viral infections (or colds) which now rarely occurred, as opposed to previous incidence of every 2-3 months.

Subject 5: Erectile dysfunction resolved within 2 weeks of starting low dose oral dipyridamole at 2 mg. The subject found that when taken with food, 2 mg was not that effective but 5 mg was. However 10 mg did not produce the same benefit as 5 mg. The subject also found that use of low dose oral dipyridamole alleviated his long-standing depression. The subject was no longer using SSRI's due to their risk of causing erectile dysfunction.

This example illustrates that low dose oral dipyridamole is effective for the treatment of erectile dysfunction and suggests that it is a viable substitute for drugs currently approved for erectile dysfunction.

### Example 8

### Treatment of Chronic Lymphocytic Leukemia

In order to determine the effect of low dose oral dipyridamole for treating chronic lymphocytic leukemia (CLL), 2 subjects with CLL were treated with low dose oral dipyridamole prepared as in Example 1 as the sole treatment.

Subject 1 (75 y/o female) and Subject 2 (74 y/o male) were treated with varying doses (2 or 3 mg) taken in the morning, afternoon or evening, for periods of up to 2 weeks for each dose and followed for up to 6 months in total.

Subject 1: 2 mg was found to be the optimal dose for lowering of the white blood cell count from 25,000 (which was rising by 10%/month on observation only) to 18,000 in a one month period. Additional effects included significantly improved energy and memory recall.

Subject 2: 3 mg was found to be the optimal dose for lowering of the white blood cell count from 30,000 to 18,000 over 6 months. Additional effects included renewed growth of eyebrows and eyelashes.

This example illustrates that low dose oral dipyridamole is effective for the treatment of CLL. The possibility of low dose oral dipyridamole in conjunction with standard treatments for CLL remains to be investigated.

### Example 9

### Prevention and Treatment of Viral Disease

In order to determine the effect of low dose oral dipyridamole for preventing and treating viral disease, 4 subjects with frequent viral infections (with upper respiratory symptoms) were treated with low dose oral dipyridamole prepared as in Example 1 or example 2 as the sole treatment.

Subject 1 (45 y/o male), Subject 2 (15 y/o male), Subject 3 (20 y/o female), and Subject 4 (33 y/o male) were treated with varying doses (2, 5 and 10 mg) taken at any time during the day, for periods of 2 weeks for each dose, and thereafter continuously for up to 2 years.

Subject 1: 2 mg was found to be the optimal dose for decreasing the frequency and duration of viral infections from 6 times yearly, lasting up to 10 days to one episode of influenza once in 2 years, lasting only 2 days and two episodes of upper respiratory tract infection yearly for 2 years, lasting only 1 day. An additional effect included vivid dreams.

Subject 2: 2 mg was found to be the optimal dose for decreasing the duration of cold symptoms from 5-7 days to 12-24 hours. An additional effect included vivid dreams.

Subject 3: 2 mg taken once daily was found to be the optimal dose for reducing incidence of viral infections from 5-6 times yearly to 1-2 times yearly. When an infection did occur, accompanied by symptoms such as sore throat, watery eyes, headache and sometimes fever, a dose of 2 mg twice daily resolved the symptoms very quickly—within 1-2 days, whereas previously these episodes would last 7-10 days. On one occasion the subject tried a 10 mg dose but this failed to deliver the same benefit as the 2 mg dose. Vast improvement in chronic depressive symptoms was also observed.

Subject 4: 2 mg was taken twice daily for a severe viral infection (upper respiratory symptoms plus viral conjunctivitis) with rapid complete relief of symptoms (in 1-2 days). Thereafter the subject continued taking 2 mg daily for 10 months without any recurrence of infections, which previously would occur every 3-4 months. Improved deep sleep and alleviation of anxiety was also reported.

This example illustrates that low dose oral dipyridamole is effective for the prevention and treatment of viral infections.

### Example 10

### Treatment of Cancer-Related Bone Pain

In order to determine the effect of low dose oral dipyridamole for treating cancer-related bone pain, a single subject with cancer-related bone pain was treated with low dose oral dipyridamole prepared as in Example 1 as the sole treatment.

A 70 y/o male was treated with 2 mg taken in the early evening hours. The subject reported resolution of bone pain related to metastatic prostate cancer to the cervical spine within 4 days. The subject was previously dependent on the daily use of opiate pain medications for the past 6 months. An additional effect observed was improved energy.

This example illustrates that low dose oral dipyridamole is effective for the treatment of cancer-related bone pain.

### Example 11

### Treatment of Chronic Rheumatic or Fibromyalgia Pain

In order to determine the effect of low dose oral dipyridamole for treating chronic rheumatic or fibromyalgia pain, 3 subjects with chronic rheumatic pain were treated with the addition of low dose oral dipyridamole prepared according to Example 1 to their present pain regimen.

Subject 1 (39 y/o female) was treated with 2 mg taken once daily with the first daily dose of oxycodone. Subject 2 (40 y/o male) was treated with 2 mg or 5 mg taken once daily, in conjunction with oxycodone. Subject 3 (45 y/o female) was treated with 2 mg, 5 mg or 10 mg taken once daily with PERCOCET.

Subject 1: After adding dipyridamole 2 mg the subject had complete relief of pain related to chronic fibromyalgia. She had previously been dependent on oxycodone 10 mg twice daily with incomplete pain relief.

Subject 2: After adding dipyridamole 2 mg the subject had complete relief of pain related to chronic rheumatic pain in the joints. When trying 10 mg instead, there was negligible pain relief 2 mg was continued for 12 months. Within 3 months of use, this subject was able to completely wean off oxycodone while maintaining pain relief.

Subject 3: After adding dipyridamole 2 mg the subject had complete relief of pain related to chronic fibromyalgia and rheumatoid arthritis. At a 5 mg dose, she experienced less pain relief, so she resumed 2 mg. She had previously been dependent on Percocet with incomplete pain relief. Percocet was slowly weaned off after 2 months of low dose dipyridamole use.

This example illustrates that low dose oral dipyridamole is effective for the treatment of chronic rheumatic and fibromyalgia pain. It also suggest that low dose oral dipyridamole can serve as a substitute for opiates in some patients.

### Example 12

### Xerostomia Treatment

In order to determine the effect of low dose oral dipyridamole for treating xerostomia, 2 subjects with xerostomia were treated with low dose oral dipyridamole prepared as in Example 1 as the sole treatment.

Subject 1 (40 y/o male) and Subject 2 (67 y/o female) were treated with varying doses (1, 2, 3, 4, 5 and 10 mg) taken in the morning or evening, for periods of 2 weeks for each dose then continued on optimal dose for 15 months in total.

Subject 1: 2 mg was found to be the optimal dose for complete resolution of xerostomia. Occasionally (once every few weeks) symptoms recurred, but only lasted 2-3 hours and were 70% less severe than before. An additional effect included improved sleep.

Subject 2: 3 mg was found to be the optimal dose for complete resolution of xerostomia (and dry eye symptoms) related to Sjogren's syndrome of 12 years duration. An additional effect included deep sleep.

This example illustrates that low dose oral dipyridamole is effective for the treatment of xerostomia.

### Example 13

### Treatment of Androgenetic Alopecia

In order to determine the effect of low dose oral dipyridamole for treating androgenetic alopecia, a single subject with androgenetic alopecia was treated with low dose oral dipyridamole prepared as in Example 1 as the sole treatment.

A 35 y/o male was treated with varying doses (2 and 4 mg) taken in the morning or evening, for periods of 2 weeks for each dose and continued with the optimal dose for 14 months in total.

A dose of 4 mg was found to be the optimal dose for expansion of hairline, thicker hair, and decreased hair loss in addition to increased hair growth of chest and abdominal hair in a subject with previously accelerated hair loss and thinning body hair which had been unresponsive to standard treatment.

This example illustrates that low dose oral dipyridamole is effective for the treatment of androgenetic alopecia. Results of this example are consistent with reports of increased hair growth in example 5.

### Example 14

### Prostate Cancer Treatment

In order to determine the effect of low dose oral dipyridamole for treating prostate cancer, a single subject with prostate cancer was treated with low dose oral dipyridamole prepared as in Example 1 as the sole treatment.

A 74 y/o male was treated with 2 mg taken in the afternoon or evening and followed for 3 months.

A daily dose of 2 mg decreased PSA (Prostate Specific Antigen) from 19.1 to 14 in a 3 month period. Additional effects included improved sleep and cognition.

This example illustrates that low dose oral dipyridamole is effective for the treatment of prostate cancer.

Examples 3-14 illustrate the surprising benefits of low dose oral dipyridamole. Contrary to expectation, the benefits are dependent on low dose and dissipate or disappear when dosing is increased above 10 mg daily.

Spontaneous patient reports of effects on dreams, dream recall, sleep, memory and cognition suggest that low dose oral dipyridamole (below 10 mg/day) operates via a different physiologic mechanism than conventional high dose oral dipyridamole therapy.

Due to the dependence of dipyridamole on stomach acidity for optimal absorption, in certain cases of drug induced or age related gastric hypo-activity, doses of 10 mg up to 20 mg may conceivably be needed to achieve the same benefit as most subjects would have with doses of 2 to 5 mg daily.

### Example 15

### Dipyridamole in Combination with Melatonin

In order to examine the sleep inducing effects of dipyridamole combined with melatonin, six subjects with treatment-refractory insomnia, unresponsive to melatonin and somewhat responsive to low dose dipyridamole (1 or 2 mg daily) were treated with a combination of dipyridamole and melatonin. The dipyridamole dose ranged between 0.5 mg and 5 mg per pill. The melatonin dose ranged between 0.25 mg and 10 mg per pill. Melatonin and dipyridamole were combined in a single oral dosage form. In all cases, subjects reported complete restoration of normal sleep after the first dose. The dose had to sometimes be reduced as the subjects felt that they were over-sleeping. The synergy between dipyridamole and melatonin is surprising and not yet understood.

1-13. (canceled)

14. A method of treating a disorder selected from the group consisting of dry eye disease, sleep disorders, nonspecific recurrent sore throat, erectile dysfunction, viral disease, pain, xerostomia and Graft-versus-Host Disease (GvHD) comprising: [symbol]
and
administering a pharmaceutical composition comprising 10 mg or less of dipyridamole and wherein said pharmaceutical composition is an oral dosage form.

15. A method according to claim 14, wherein the oral dosage form comprises 0.125 mg, 0.25 mg, 0.5 mg, 1 mg, 2 mg, 2.5 mg, 3 mg, 4 mg, 5 mg, 6 mg, 7 mg, 7.5 mg, 8 mg, 9 mg or 10 mg of dipyridamole.

16. A method according to claim 14, wherein the oral dosage forms comprises 1 mg, 2 mg, 3 mg or 5 mg of dipyridamole.

17. A method according to claim 14, wherein the disorder is dry eye disease.

18. A method according to claim 14, wherein the disorder is a sleep disorder.

19. A method according to claim 18, wherein the sleep disorder is insomnia.

20. A method according to claim 19, wherein the insomnia is treatment-refractory insomnia.

21. A method according to claim 14, wherein the disorder is a nonspecific recurrent sore throat.

22. A method according to claim 14, wherein the disorder is erectile dysfunction.

23. A method according to claim 14, wherein the disorder is viral disease.

24. A method according to claim 14, wherein the disorder is xerostomia.

25. A method according to claim 14, wherein the disorder is pain.

26. A method according to claim 14, wherein the pain is rheumatic or fibromyalgia pain.

27. A method according to claim 14, wherein the pain is angina pectoris.

28. A method according to claim 14, wherein the pain is cancer-related bone pain.

29. A method according to claim 14, wherein the daily dose of dipyridamole is 10 mg or less.

30. A method according to claim 14, wherein the oral dosage form further comprises melatonin.

31. A method according to claim 30, wherein the oral dosage form comprises between 0.25 mg and 10 mg of melatonin.

32. A method according to claim 30, wherein the disorder is a sleep disorder.

33-42. (canceled)
